(19) United States
(12) Patent Application Publication
(10) Pub. No.: US 2023/0119680 A1
(43) Pub. Date: Apr. 20, 2023

(54) MACHINE LEARNING SYSTEM FOR, AND METHOD OF ASSESSING AND GUIDING MYOCARDIAL TISSUE ABLATION AND ELIMINATION OF ARRHYTHMIA

(71) Applicants: Patrick Maguire, Foster City, CA (US); Dena Maguire, Foster City, CA (US)

(72) Inventors: Patrick Maguire, Foster City, CA (US); Dena Maguire, Foster City, CA (US)

(21) Appl. No.: 17/963,895

(22) Filed: Oct. 11, 2022

Related U.S. Application Data
(60) Provisional application No. 63/360,593, filed on Oct. 18, 2021.

(51) Int. Cl. A61B 18/14; A61B 5/055

(52) U.S. Cl. CPC A61B 18/1492; A61B 5/055; A61B 2018/00577

(57) ABSTRACT

A machine learning system for evaluating at least one characteristic of myocardial tissue and its ablation or subset thereof, which includes a training mode and a production mode. The training mode is configured to train, assess and guide a computer and construct a transformation function to predict an anatomical, physiologic, electric, metabolomic, or genetic manifestation leading to alterations, including ablation, that predict and unknown structural or functional characteristic of myocardial tissue and a subsequent aberration that results in abnormal electrical signal and subsequently results in abnormal heart function. The production mode is programmed to use any transformational function to predict the unknown electroanatomic and metabolic characteristic that result in arrhythmia and abnormal myocardial function and guide subsequent ablation and elimination of arrhythmogenic foci.

### CROSS-REFERENCE TO RELATED APPLICATION(S)

This application claims priority under 35 U.S.C. § 119(e) of the U.S. Provisional Patent Application Ser. No. 63/360,593, filed Oct. 18, 2021 and titled, “MACHINE LEARNING SYSTEM FOR ASSESSING AND GUIDING MYOCARDIAL TISSUE FOR SUBSEQUENT ABLATION AND ELIMINATION OF ARRHYTHMIA.,” which are hereby incorporated by reference in their entirety for all purposes.

### FIELD OF INVENTION

The present disclosure relates generally to the fields of machine learning, computer modeling, simulation and computer aided design. More specifically the disclosure relates to computer-based machine learning and systems and methods for constructing and executing models of cardiac anatomy, physiology and imaging that the preselected ablation to be used to guide non-invasive cardiac ablation and ultimately improve efficacy.

### BACKGROUND OF THE INVENTION

Cardiovascular disease is the leading cause of death in the United States and claims 600,000 lives annually. According to the American Heart Association, more than three million Americans are diagnosed with an abnormal condition of heart electrical conduction.

### SUMMARY OF THE INVENTION

Therapeutic options for treatment to establish normal cardiac electrophysiology include medications and various means to apply energy to abnormal tissue and electrical pathways that are responsible for abnormal cardiac function.

Embodiments according to the present disclosure related to the application and use of machine learning to guide an arrhythmia ablation plan to remove the arrhythmia. Metrics or inputs to the machine learning assessment are obtained and allow optimization of a treatment target.

The proper assessment and diagnosis of cardiac electrical operation is essential for ensuring high quality care and resumption of normal cardiac contractility and function. Several imaging and electroanatomic modalities are used to inspect the condition and function of the cardiac electrical system and its functional sequelae. Various radiologic imaging technologies such as computed tomography (CT), magnetic resonance imaging (MRI) and ultrasound are used in some embodiments. Electroanatomic assessment of various regions of the heart that can demonstrate normal or abnormal electrical conduction may be assessed and can correlate with imaging to guide location of ablation.

Imaging and electrical modalities have strengths and weaknesses that limit their ability to provide a complete and comprehensive assessment of electrical and structural assessment of the abnormal electrical conduction that can lead to a dysfunctional heart result. In some embodiments, machine leaning is used to combine all of the data, imaging, anatomy, electrophysiology and subsequent permutations, which lead to more specific targets of abnormal electrical function that is susceptible to ablation.

Patients diagnosed with arrhythmia that are clinically significant and result in symptom can be candidates for an ablative procedure to rid the patients' arrhythmia. An accurate understanding of the arrhythmia and it resultant changes in overall heart function, and perhaps ablation is essential to a favorable outcome and enhanced efficacy.

Methods to assess the arrhythmia abnormality and the ability to combine imaging and metabolic and electrical data make it possible to more accurately predict the success of upcoming therapeutic interventions of the arrhythmia. Such technology needs to provide clear and demonstrable benefits to lessen or ablate the arrhythmogenic tissue and restore normal myocardial function. Technologies should not expose, reduce, or avoid patients to excessive medical risk, should be cost effective and should be shown to have a favorable benefit-risk profile.

To improved diagnostic and treatment capabilities it is desired to have a system for quick and accurate assessment of cardiac images, electroanatomic results, metabolic profile all that may be synthesized and merged to allow a machine learning approach to accurate delineation of ablation volume treated, preferably with non-invasive means using cardiac radiosurgery. Enhanced and accurate targeting of anatomic arrhythmia foci and surrounding tissue can lead to a more precise and accurate ablation and sparing of otherwise normal tissue that need not be ablated to preserve ventricular function. In other words, avoiding or preventing ablating normal/healthy tissues are also within the scope of the Present Disclosure.

Computer modeling and machine learning can be performed to analyze location of arrhythmogenic sites for ablation. Ablation targets can be anatomically defined, structurally defined based on motion or contractility, functionally defined based on presence or aberrance of electrical signals, or even metabolically defined based on percentage of scared myocardium and its inherent electrical membrane channels to accurate define the target. Such scans and data can be merged to provide an opportunity for machine learning and predictive modeling of the ablation target location, and subsequently predict the success of the ablation, based on volume of tissue treated, dose etc.

Computer simulation and modeling has helped and predictive capability to evaluate the electrophysiologic signals and have not taken advantage of tissue voltage maps, CT scans, electroanatomic maps, MRI scans and metabolic maps from the MRI merged to give a predictive composite anatomy map and target. Once an accurate ablation target with its characteristics has been identified. Data can be accumulated to associate treatment and imaging metrics with an ablation outcome. Predictive ablation outcomes can be tested against permutations in variable such as volume treated, (e.g., a specific volume having a certain voltage signal.)

The machine learning system and method described in this disclosure facilitates the identification of myocardial targets and treatment of electrophysiologic disease of the heart. The system and the method facilitate the evaluation, treatment planning and assessment of myocardial tissue to be ablated. Imaging and function studies that are electrophysiologic or metabolic in nature and two-, three- or four-dimensional imaging studies and the use of machine learning system can also incorporate hemodynamic and voltage data, which can be combined singularly or in plural to describe and draw, and are of myocardial tissue or structures that are responsible for, and then can be ablated to eliminate hazardous arrhythmia.

Referring to FIG. 2A, which illustrates a machine learning system in accordance with some embodiments. The machine learning system for evaluating one characteristic of the heart and its electrophysiologic properties and aberrancies, which include a training mode and a production mode. The training mode is configured to train a computer and construct a transformation function to predict aberrant anatomy and electrophysiologic conduction, which is responsible for the arrhythmia. Such abnormal electrophysiologic can result in functional abnormalities of contractile, valvular or other cardiac abnormalities that increase morbidity and mortality.

Referring to FIG. 3, which illustrates a training mode and a production mode of the machine learning system in accordance with some embodiments. The training mode is configured to compute and store in a feature vector the known characteristic, be it from imaging, catheter, or other data. The training mode is further configured to store data form all imaging and electrophysiologic sources to store data associated with abnormal conduction that results in arrhythmia. The training mode is perturbed at least one anatomic or electrophysiologic characteristic associated with the location, genesis, and propagation of abnormal cardiac rhythm. The training mode is then to calculate, and demonstrate volumes associated with abnormal cardiac rhythm. In some embodiments, the training mode is further configured to repeat a set of perturbations and calculate and store steps to create a feature vectors and volume, and to generate the transformation function.

In some embodiments, the training mode is further configured to perform the function involving the patient-specific data and thereby generating a digital model of at least one volume and its electrophysiologic properties; discretizing the digital model; applying boundary conditions for the volume of cardiac tissue responsible for the arrhythmia of the digital model; and initializing a solving a mathematical equation to describe the volume and radiation dose required to ablate the predetermined volume of myocardial.

In some embodiments, the method includes storing quantities and parameters that anatomy of the myocardium, and electrophysiologic state of the ablation volume of interest. In some embodiments, the method further includes perturbing and anatomic parameter, an electrophysiologic parameter that characterized the digital image that is created. In another embodiment, the method includes further re-discretizing and/or solving the mathematical equations with the physiologic and anatomic parameter that are perturbed together or singularly. The embodiment further stores quantities and parameters.

Still referring to FIG. 3, which illustrates a production mode that is configured to receive one or, more feature vectors. In some embodiments, the production mode is configured to apply the transformation function to the feature vectors. In some embodiments, the production mode is configured to store quantities of interest. In some embodiments, the production mode is configured to process the quantities of interest to provide data for use in at least one evaluation, diagnosis, prognosis, treatment, treatment planning related to the heart, the myocardial tissue contained therein and electrical conduction system residing in such heart. Such parameter, quantities, volumes, images, tracings, signals, treatment plans, beam data, RTP files, etc. are able to be stored, recalled, and generate statistical features, trends and predictive features.

In one aspect, a computer implemented machine learning method for evaluating at least one characteristic of the arrhythmia and myocardial volume may involve training a computer by using a training mode of a machine learning system to construct a transformation function to predict an and unknown anatomic or electrophysiologic characteristic of myocardial tissue using a known electrophysiologic or anatomic characteristic. The method may involve using a production mode of the machine learning system to direct the transformation function to predict the unknown anatomic or electrophysiologic. This may be expanded to machine learning to describe, discretize, predict, using dose plans the expected electrophysiologic and anatomic result that leads to ablation of the arrhythmia.

In another aspect, the method further includes using the training mode to compute and store in a feature vector the know anatomic or electrophysiologic characteristic of that volume of myocardial tissue. In some embodiments, the method further includes using the training mode to store quantities associated with the electrophysiologic state. In some embodiments, the method further includes using the training mode to calculate and to perturb the known electrophysiologic and anatomic characteristics of the heart is question stored in the feature vector. In some embodiments, the method further includes using the training mode to calculate a new ablation target with the perturbed known anatomic or electrophysiologic characteristic using various interactions and changes in model parameters, (e.g. volume treated, amount of epicardium, myocardium and endocardium treated). In some embodiments, the method further includes using the training mode to store quantities associated with the new ablation target through the perturbed characteristic or variable. In some embodiments, the method further includes using the training mode to repeat perturbing, calculating and storing steps to create a set of feature vectors and volume vectors to generate a transforming function.

In some embodiments, the present disclosure is used in anatomic modeling study, in-vitro structural issue predictions and corrections, and/or bioengineering applications, which do not involves actual surgical procedures and/or medical treatments.

### BRIEF DESCRIPTION OF THE DRAWINGS

FIG. 1 illustrates localizing the cardiac volume and a method of performing a cardiac ablation in the lower ventricular septum on a predetermined site in accordance with some embodiments;

FIG. 2A is a block diagram that illustrates a training mode and a production mode of the machine learning system in accordance with some embodiments;

FIG. 2B illustrates a mesh structure describing a heart in length, width and depth with area of susceptible to arrhythmia for radioablation in accordance with some embodiments;

FIG. 3 is a flowchart of a computer implemented method for generating a pattern for ablation based on machine learning inputs in accordance with some embodiments;

FIG. 4 is a block diagram that illustrates a computer system in accordance with some embodiments;

FIG. 5 is a block diagram of a basic software system that is employed for controlling the operation of computing device in accordance with some embodiments; and

FIG. 6 illustrates a diagnostic and treatment system in accordance with some embodiments.

### DETAILED DESCRIPTION OF THE INVENTION

This disclosure describes machine learning systems and methods that qualitatively and quantitatively characterize anatomic geometry and electrophysiology of the heart with respect to normal function and location of arrhythmogenic disturbance. Reference may be made to characterizing or evaluating the heart and its electrophysiologic pattern, especially the arrhythmia. In some embodiments, such characterization for evaluation is able to be performed on a heart and the electrophysiologic signal and aberrant signals that are characterized as arrhythmias. The various embodiments described herein is able to be applied to any heart (e.g., any organs, such as animal hearts and human hearts, or biological cells), surface or structure and/or combinations of heart structures. Illustrations of the systems and methods via example is not intended to limit the scope of the computer modeling and simulation systems and methods describe herein.

Reference is made in detail to the embodiments of the present disclosure, examples of which are illustrated in the accompanying drawings. While the invention is described in conjunction with the embodiments below, it is understood that they are not intended to limit the present disclosure to these embodiments and examples. On the contrary, the present disclosure is intended to cover alternatives, modifications and equivalents, which can be included within the spirit and scope of the invention as defined by the appended claims. Furthermore, in the following detailed description of the present disclosure, numerous specific details are set forth in order to more fully illustrate the present disclosure. However, it is apparent to one of ordinary skill in the art having the benefit of this disclosure that the present invention can be practiced without these specific details. In other instances, well-known methods and procedures, components and processes have not been described in detail so as not to unnecessarily obscure aspects of the present invention. It is, of course, appreciated that in the development of any such actual implementation, numerous implementation-specific decisions are made in order to achieve the developer's specific goals, such as compliance with application and business-related constraints, and that these specific goals vary from one implementation to another and from one developer to another. Moreover, it is appreciated that such a development effort can be complex and time-consuming, but is nevertheless a routine undertaking of engineering for those of ordinary skill in the art having the benefit of this disclosure.

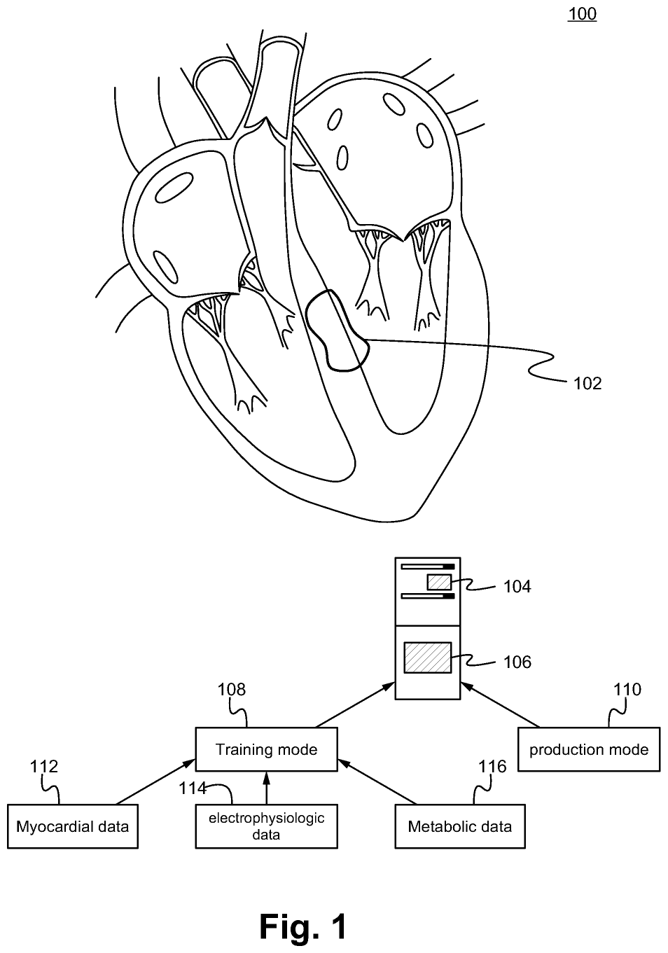

FIG. 1 illustrates a method 100 of performing a cardiac ablation in the lower ventricular septum on a predetermined site 102. In some embodiments, the machine learning system is used, which includes two modes: a training mode and a production mode. The two modes are embodied in a computer system 104 and with computer readable medium 106.

In some embodiments, the system executes the two modes in a series, where the training mode 108 is executed first, and the production mode 110 is executed second. In other embodiments, the production mode 110 is executed before the training mode 108, such as by using a pre-stored data or model obtained from other patients or computers. A person of ordinary skilled in the art appreciates that any other performing orders or repetitions are within the scope of the present disclosure.

The training modes 108 is executed to develop analytical capabilities in the computer system 104 that enables the computer system 104 to predict unknown anatomic or electrophysiologic characteristics of a myocardial volume. These predictive capabilities are developed by the analysis and or evaluation of to the known myocardial volume. Upon a collection of known anatomic or electrophysiologic characteristics, the computer 104 is trained/programmed to predict various unknown anatomic, myocardial volume and/or electrophysiologic characteristics. The abstract mapping that transforms a set of known characteristics are referred to as “transformation function.” In some embodiments, the training mode 108 is configured to construct the transformation function.

In other embodiments, the production mode 110 of the machine learning system uses a transformation function to predict the myocardial volume or electrophysiologic characteristics that are unknown from a collection of myocardial and electrophysiologic or metabolic characteristics that are known. Hence, during execution of the production mode 110, input into the transformation function includes a set of known myocardial, electrophysiologic or metabolic characteristics used during the training mode 108. In other embodiments, the output of the transformation function is one or more myocardial volumes and or electrophysiologic characteristics that are previously unknown and that contains or lacks abnormal myocardial tissue that is related to arrhythmia. In some embodiments, the transformation function is designed to accommodate the data of a specific vector, such that more than one types of data (image, volume, voltage for example) can be more easily compared, which facilitates learning and algorithm development. Further, changes in images and electrical signals and data when inserted into the algorithm can influence the characterization of the arrhythmia location for ablation.

In other embodiments, the training mode 108 and production mode 110 are implemented in a number of different ways in various alternative embodiments. One embodiment of a method for implementing the training mode 108 and a production mode 110 of a machine learning system is described in more detail below. This is one of the exemplary embodiments, however, and should not be interpreted as limiting the scope of machine learning system as described above.

During the training mode 108 of the machine learning system, a myocardial data 112, electrophysiologic data 114, metabolic data 116, or a combination thereof is acquired that characterizes the state and operation of a myocardial volume and its electrophysiologic characteristics. These data are collected through one or more acquisition methods, including for example analysis of radiologic images, analysis of echocardiographic images, analysis of electroanatomic images and maps, electrophysiologic signals and arrhythmia tracings clinical instruments (e.g., sensors, blood pressure gauges), metabolic signals from magnetic resonance, images from positron emission tomographic images, and computer modeling/simulation.

Referring to a myocardial volume containing cellular characteristics that initiate, slow, block, interfere with, accelerate and electrical signal, such parameters include, for example, myocardial depth, width, volume, motion characteristic in dimensions and are adjacent to structures, cavities or other structures nearby (vasculature).

In some embodiments, the parameters and factors for ablation to be considered also include approximations to size, volume and location, myocardial electrical signals, block and progression of signals, aberrant or normal, surrounding vasculature, e.g., diameter, eccentricity, cross-sectional area, axial length, length of major or minor axis of the myocardial tissue or segment via simplified and or analytic model, which describes these variables including height shape, lateral profile thickness, degree of calcification, angular size, radial length, rigidity, flexibility, movement, tissue properties, attachments or proximity to other structures.

In some further embodiments, the parameters and factors for ablation to be considered also include size shape density composition, extent of abnormality or calcification, relationship to coronary arteries and veins and valves, which are all considerations and are considered organs at risk for treatment planning of ablation procedures.

In some embodiments, the parameters and factors for ablation to be considered include stroke volume of the chamber, and/or cardiac output that is calculated, blood pressure, heart rate, ejection fraction, weight, body mass index, race or gender of the patient. In addition to the location, duration, extent, power, among other factors for an effective ablation treatment, risk avoidance (e.g., damage or risk of damage to the subject organs or tissues) using the above factors are also part of the considerations of the present disclosure.

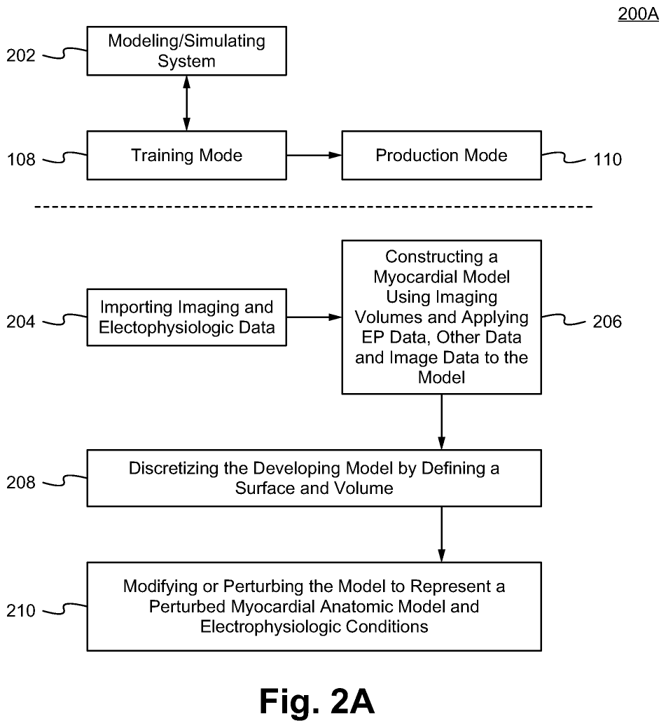

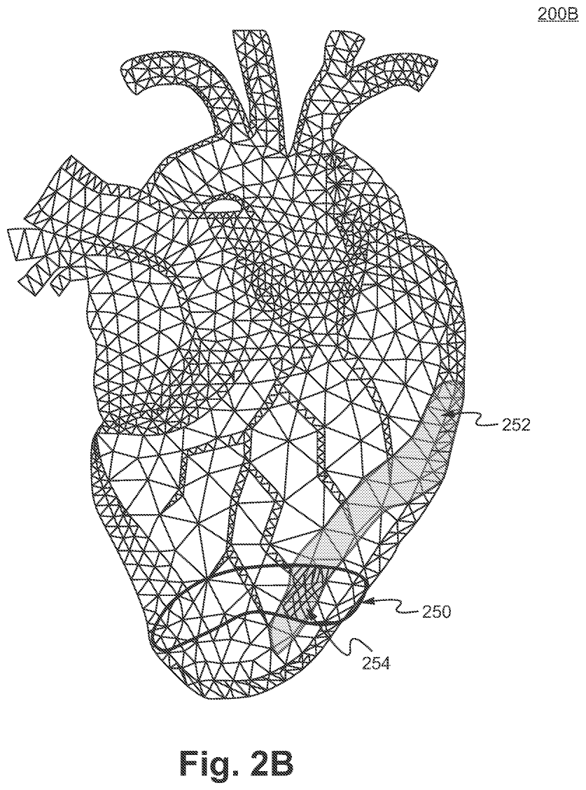

FIG. 2A illustrates machine learning method 200A having the training mode and the production mode in accordance with some embodiments. FIGS. 1 and 2 can be read together, wherein similar referencing numbers can refer to the same or similar functions or structures. The method 200A implements the training mode 108 of the machine learning system 100. In this embodiment, the training mode 108 of the machine learning system 100 is coupled with a modeling or a simulation system 202, which provides input data for the machine learning system 100. Hence, the modeling and simulation system 202 operates in conjunction with the machine learning system 100 in that it provides myocardial data 112 (e.g., myocardial volume) and electrophysiologic data 114 or metabolic data 116 to the machine learning system 100. These data serve as the foundation from which the machine learning system 100 learns to perform the predetermined task(s) that are reflective of a dose, volume and target for arrhythmia ablation.

In some embodiments, the method 200A is performing the following steps. At a Step 204, patient-specific geometric, anatomic, electrophysiologic and other data from a computer system are imported. At a Step 206, a (possible parameterized) model using the imported data is constructed. At a Step 208, a developing model by defining a surface and volume is discretized.

A myocardial model construction of the Step 206 is further illustrated blow. In some embodiments, the geometric model of the myocardial model contains a multidimensional digital representation of the relevant patient anatomy, which includes a myocardial volume optimal for precise arrhythmia ablation and one aspect of an electrophysiologic model. In some embodiments, the model also includes one or more sections of the heart and its internal electrical normal or abnormal data. In some other embodiments, the model is created using imaging data and at least one clinical measured electrophysiologic signal parameter such as voltage signal. In some embodiments, imaging data is obtained for any suitable diagnostic imaging exams such as those listed above including electroanatomic mapping of electrical signals and arrhythmias. Clinically measure data parameters are obtained from the suitable tests such as those listed above.

In some embodiments, a data map is obtained from an electrophysiologic voltage study, maps the voltage, the electrical signals, and subsequently the tissue voltage. Low voltage is indicative of myocardial tissue that is infiltrated with scar and thus a nidus for development of ventricular arrhythmia. A ‘voltage map’ can be fused to or combined with a cardiac gated CT scan. In some embodiments, areas of voltage at 0.5 mV or less can be contoured as a presumed area of tissue likely to lead to arrhythmia. This myocardial 3-D volume can be treated with a dose of energy (25 Gy or greater) to suppress, block, and/or eliminate arrhythmia. Different sections of the heart have different electrophysiologic signals that can be distinguished as a different signal and voltage, which are used as criteria for prediction, diagnostic, and treatment in some embodiments. For example, diminution in electrical signal can be indicative of possible changes in tissue morphology, such as scar.

In some embodiments, late gadolinium enhanced cardiovascular magnetic resonance imaging (LGE-CMR), and the volume of tissue that this encompasses, can be related to the volume of tissue that causes ventricular tachycardia. The direct area of scar is visualized with gadolinium enhancement. The “shadow or penumbra” of tissue containing scar, fibroblasts and ischemic tissue can be responsible for ventricular arrhythmia development. This quantifiable image of ‘penumbra’ is able to be fused to the cardiac gated CT scan as an input/factor to the development of the arrhythmia. Patients with a percentage increase (10% or greater) of the percentage of heart tissue that has this penumbra, or shadow, is known to lead to ventricular arrhythmia. This can be another input to the machine model.

In some embodiments, a digital anatomic model is created using applied mathematics and image analysis, not limited to image segmentation, machine learning, computer aided design, parametric curve fitting and polynomial approximation. In some embodiments, a hybrid approach that combines modeling techniques is used. A final multi-dimensional model provides a digital surrogate that captures the relevant physical features of the myocardial topology under consideration and may contain one or more morphological simplification that exploit underlying myocardial geometric features of a patient-specific myocardial volume being considered for ablation or alteration to treat the arrhythmia.

Now referring to the Step 208, following the construction of the digital model as described above, the modeling and simulation portion of the machine learning system discretize the surface and volume of the myocardial tissue model into a fmite number of partitions. These individual and non-overlapping partitions, called “elements” facilitate the application and solution of the myocardial volume model that contains the myocardial tissue of interest. The set of surface and volume elements used to discretize the model, collectively referred to as the ‘mesh’ transform the continuous geometric model into a set of mesh points and edges where each element point in the mesh has discrete w, y, and z spatial coordinates, and each element edge is bounded by two mesh points and has a finite length, which is further illustrated in the FIG. 2B.

FIG. 2B is a diagram that illustrates a mesh structure describing a heart in length, width and depth with area of susceptible to arrhythmia for radioablation in accordance with some embodiments. FIG. 2B can be read together with FIG. 2A.

Referring to the FIG. 2B, a representative mesh 200B discretizes the surface of a geometric model that outlines the tissue geometry for ablation. The geometric model in this embodiment includes a myocardial volume that contains scar or other aberrant electrophysiologic abnormalities.

As shown in the FIG. 2B, the first line area 250 identifies and area of/next to area of fibrosis. This is an area susceptible to arrhythmia generation. The second area 252 defines and area of late activation, which can be a trigger for arrhythmia. The area of intersection of these two volumes can be a target 254 for radioablation.

The shape of the surface elements and internal structural elements created by the modeling and simulation portion of the machine learning system take a form of a geometric like structure. A volume element is created by modeling and simulation systems. The surface and volume are configured into a mesh, which determines the spatial resolution of the discrete model, and can vary in space and time. The local densities of the surface and volume meshes which determines the spatial resolution the discrete model vary in space and time.

In some embodiments, the local densities of the surface and volume depend on the complexities of the local topology of the underlying geometric volume model; more complex local topology needs higher spatial resolution and therefore a higher mesh density to resolve local regions of complex topology that describes a myocardial volume, which has a dose (ablation) and volume that become a target for ablation. The modeling and simulation portion of the machine learning method can use the electroanatomic parameters to further characterize the mesh and model.

As a next step in the modeling and simulation mode of the machine learning method, a boundary condition is applied to discrete patient model. Boundary conditions can be obtained from patient-specific measurements, imaging, and other electroanatomic parameters.

A myocardial volume and electrophysiologic quantities of interest are computed by the modeling and simulation system, which may be a component of the training mode of the machine learning system.

A constructed treatment plan that identifies a dose and volume that describes the target can be fused to other images and these can be components of the training mode of the machine learning system. A plurality of treatment plans can be integrated into the model and can become a quantity of interest.

Following the computation of the quantities of interest and anatomic and electrophysiologic parameters that are inputs in the modeling and simulation systems, collectively referred to as “features” which are assembled into a vector. The vector of myocardial anatomic and electrophysiologic parameters is referred to as “feature vectors.” An illustrative example as numerical quantities contained in a feature vector include some or all of the parameters described above. Corresponding quantities of interest are computed form the simulation from a myocardial anatomic model that are characterized by a feature vector and are assembled into a vector, which is referred to as the “quantity of interest vector.” Both the feature and quantity of interest vectors are then saved for used during the other steps of the machine learning process. In addition, there are entries within the feature and quantity of interest vectors that are obtained from different mechanisms, data, simulations, etc. Nonetheless, each feature vector is associated with a quantity of interest vector and vice versa.

Referring back to the FIG. 2A, next steps in the method includes a Step 210 for modifying or perturbing the digital model and to represent a perturbed myocardial anatomic model and electrophysiologic conditions. An example of one myocardial anatomic perturbation includes a decrease in thickness of the myocardial wall during specific time in the cardiac cycle. An example of an electrophysiologic perturbation is a prolonged interval of the cardiac cycle and the development of a premature ventricular contraction.

As illustrated in the FIG. 2A, following modification in myocardial anatomic and electrophysiologic condition, the modeling and simulation portion of the machine learning system is repeated until a desired number of feature vectors and the corresponding quantities of interest vectors are obtained. Note that each iteration of the repeated process produces a new feature vector and a new quantity of interest vector. Though one or more entities with the feature and/or quantity of interest vector can change with each iteration of the repeated process, and the representation of each vector remains the same. That is, each digital model is represented by the same characteristic and the same number of characteristics and this collection of characteristics is obtained within each feature vector. The corresponding quantities of interest for each digital model are the same. The sets of feature and quantity of interest vectors are stored on digital media.

In some embodiments, and electrophysiologic perturbation is made in an area of interest that contains a volume, and prescribes a planned ablation dose. Assuming appropriate and effective dose and volume constraints are met, then a myocardial volume can rid of the arrhythmia.

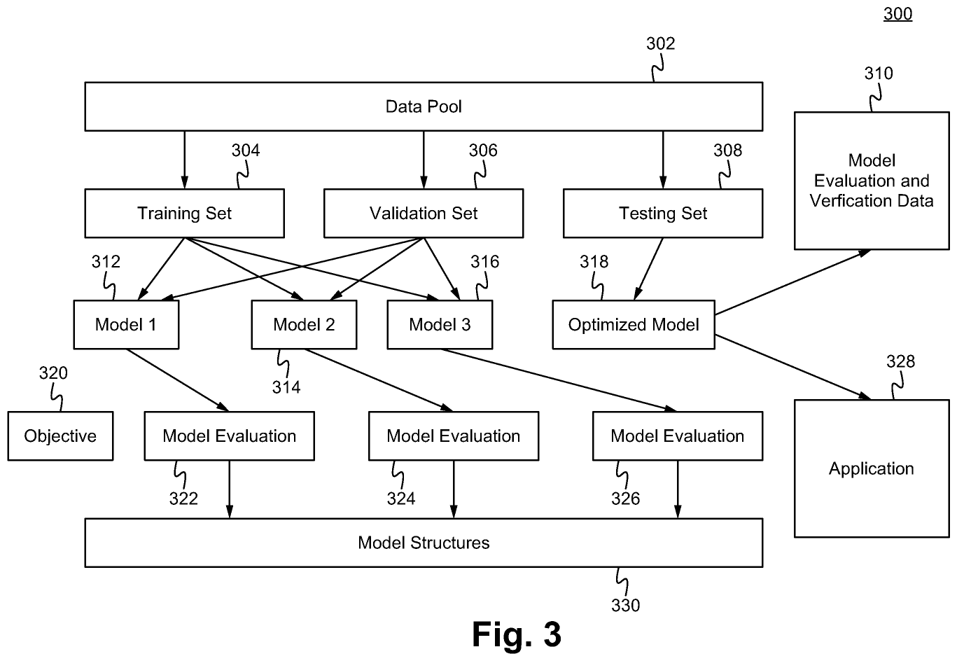

FIG. 3 illustrates a training mode and a production mode of the machine learning system 300 in accordance with some embodiments. The machine learning system 300 uses a method applying machine learning algorithms to a collection of features and quantity of interest vectors from the method described above and is illustrated in the FIGS. 2A and 2B.

In some embodiments, the data pool 302 provides data to a training set 304, a validation set 306, and a testing set 308. The output from the training set 304 and the validation set 306 are provided to a first model 312, a second model 314, and a third model 316. Each of the outputs of the first model 312, the second model 314, and the third model 316 are provided for model evaluation 322, 324, and 326 respectively. The output evaluation is then provided to create model structures 330. The output from the testing set 308 is provided to an optimized model 318, which is provided to a model evaluation and verification 310 and an application 328.

In some embodiments, the collection of features and quantity of interest vectors is first imported into machine learning software. The machine learning software then applies one or more analysis or machine learning algorithms (e.g., decision trees, support vector machines, regression, Bayesian networked, random forests) to the set of features and quantity of interest vectors. Following the application of machine learning algorithm, a transformation function is constructed. The transformation function is served as a mapping between one or more features contained withing a feature vector and the one or more quantities of interest computed from the modeling and simulation portion of the machine learning system 300. Hence, the input into the transformation function is a feature vector and the output of the transformation function is a quantity of interest vector. To test the accuracy of the transformation function created by the machine learning algorithm, for example, one of the feature vectors used to create the transformation function is used as input into the transformation function. The expected output from the transformation function is the corresponding quantity of interest vector, though the quantity of interest output vector may not be reproduced exactly by the transformation function. In some embodiments, the transformation function is stored on digital media for use during the production mode of the machine learning system.

Following construction of the transformation function by the analysis and machine learning algorithm(s), functioning of the training mode of the machine learning system as described in the present embodiment, is completed. Subsequently, the transformation function is used in the production mode of the machine learning system 300.

In some embodiments, the production mode of the machine learning system 300 is able to be used after the training mode. The production mode is configured to compute the quantity of interest vectors rapidly and accurately by applying the transformation function to a variety of feature vectors. In some embodiments, these feature vectors are used to construct the transformation function.

In some embodiments, the production mode of the machine learning system is first used to import the transformation function and one of the more feature vectors, which contain the same set of features used during the training mode. In some embodiments, the feature vectors used during the production mode is used or, in alternative embodiments, not to be used during the training mode to construct the transformation function, and therefore the transformation function may not have been constructed with the data contained within the feature vectors. The number of features within each feature vector and the quantities represented by each feature with each feature vector, however, are able to be the same as those used to construct the transformation function.

The transformation function is then applied to more for feature vectors. Hence, the inputs to the transformation functions during the production mode for the machine learning system is able to be one or more feature vectors, and the output from the transformation can be a vector that contains the quantities of interest. The quantity of interest vector outputted from the transformation function can then be stored (e.g., on digital media).

The quantities of interest contained within the quantity of interest vector can include qualitative and or quantitative geometric and electrophysiologic information. These data are further analyzed and assessed through various mechanisms of post-processing to reveal patient specific myocardial anatomic and/or electrophysiologic that aids in the diagnosis, treatment and or treatment planning of the patient to treat and ablate the problematic, such arrhythmia in the heart for such patient. The qualitative and quantitative data is used to guide clinical decision making and or provide predictive information about disease and arrhythmia progression and risk stratification of myocardial function that is affected adversely by the arrhythmia.

Quantities of interest and or data derived from the machine learning system can be delivered to physicians and for them to use these data for clinical decision-making. Delivery of patient-specific information to physicians can occur via integrated or stand-alone software systems, numerical data, graphs, plots, electronic media or combination thereof. These data are then used by an individual physician or team of physicians to develop a complete, comprehensive and accurate understanding of patient cardiac and electrophysiologic health and to determine whether or not medical treatment including an ablation of arrhythmia is warranted. When medical treatment is warranted, results from the machine learning system are used to guide clinical decision making. By way of example specific ways in which the output from the machine learning system is incorporated into the clinical management of the electrophysiologic situation, which includes potentially refractory arrhythmia that includes: analysis of the heart rhythm, its aberrancy, including diagnosing the severity, functional significance and clinical response to abnormal cardiac function secondary to arrhythmia. In the event of arrhythmia recurrence, the machine algorithm is produced again with the new data, and an appropriate clinical ablation plan containing dose and volume are reconfigured.

Patient specific selection, need for ablation, energy level to be used to accomplish the ablation, including pulse field ablation, machine learning guidance to confirm location of dose of radiation and volume of myocardial tissue to be ablated, guidance to outline the amount of unstable myocardium with a prediction to contribute to, or become arrhythmogenic, the amount of myocardial tissue to be spared and the amount of myocardial tissue to be ablated that limits the exact volume the size of volume to be ablated. The list of applications outlined above is for example purposes only, and the list is not intended to be exhaustive. The machine learning system provides a fast and accurate virtual framework for constructing patient-specific sensitivity analyses. Such analyses assess the relative impacts of myocardial geometric and electrophysiologic and cardiac function of the patient; these changes are then be assessed for functionality.

Hardware Aspect

According to some embodiments, the technical techniques described herein are implemented by at least one computing device. The techniques may be implemented in whole or in part using a combination of at least one server computer and/or other computing devices that are coupled using a network, such as a packet data network. The computing devices may be hard-wired to perform the techniques or may include digital electronic devices such as at least one application-specific integrated circuit (ASIC) or field programmable gate array (FPGA) that is persistently programmed to perform the techniques or may include at least one general purpose hardware processor programmed to perform the techniques pursuant to program instructions in firmware, memory, other storage, or a combination. Such computing devices may also combine custom hard-wired logic, ASICs, or FPGAs with custom programming to accomplish the described techniques. The computing devices may be server computers, workstations, personal computers, portable computer systems, handheld devices, mobile computing devices, wearable devices, body mounted or implantable devices, smartphones, smart appliances, internetworking devices, autonomous or semi-autonomous devices such as robots or unmanned ground or aerial vehicles, any other electronic device that incorporates hard-wired and/or program logic to implement the described techniques, one or more virtual computing machines or instances in a data center, and/or a network of server computers and/or personal computers.

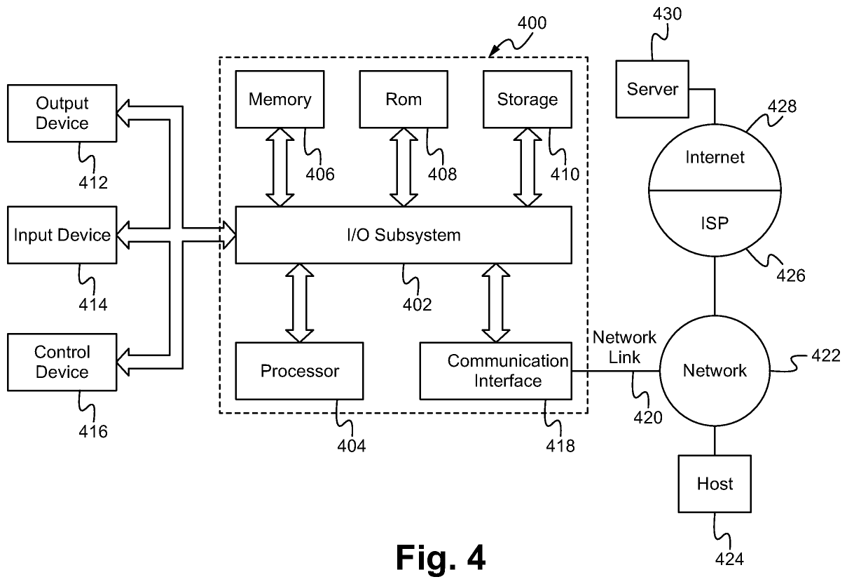

FIG. 4 is a block diagram that illustrates an example computer system in accordance with some embodiments. In the example of FIG. 4, a computer system 400 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

Computer system 400 includes an input/output (I/O) subsystem 402 which may include a bus and/or other communication mechanism(s) for communicating information and/or instructions between the components of the computer system 400 over electronic signal paths. The I/O subsystem 402 may include an I/O controller, a memory controller and at least one I/O port. The electronic signal paths are represented schematically in the drawings, for example as lines, unidirectional arrows, or bidirectional arrows.

At least one hardware processor 404 is coupled to I/O subsystem 402 for processing information and instructions. Hardware processor 404 may include, for example, a general-purpose microprocessor or microcontroller and/or a special-purpose microprocessor such as an embedded system or a graphics processing unit (GPU) or a digital signal processor or ARM processor. Processor 404 may comprise an integrated arithmetic logic unit (ALU) or may be coupled to a separate ALU.

Computer system 400 includes one or more units of memory 406, such as a main memory, which is coupled to I/O subsystem 402 for electronically digitally storing data and instructions to be executed by processor 404. Memory 406 may include volatile memory such as various forms of random-access memory (RAM) or other dynamic storage device. Memory 406 also may be used for storing temporary variables or other intermediate information during execution of instructions to be executed by processor 404. Such instructions, when stored in non-transitory computer-readable storage media accessible to processor 404, can render computer system 400 into a special-purpose machine that is customized to perform the operations specified in the instructions.

Computer system 400 further includes non-volatile memory such as read only memory (ROM) 408 or other static storage device coupled to I/O subsystem 402 for storing information and instructions for processor 404. The ROM 408 may include various forms of programmable ROM (PROM) such as erasable PROM (EPROM) or electrically erasable PROM (EEPROM). A unit of persistent storage 410 may include various forms of non-volatile RAM (NVRAM), such as FLASH memory, or solid-state storage, magnetic disk, or optical disk such as CD-ROM or DVD-ROM and may be coupled to I/O subsystem 402 for storing information and instructions. Storage 410 is an example of a non-transitory computer-readable medium that may be used to store instructions and data which when executed by the processor 404 cause performing computer-implemented methods to execute the techniques herein.

The instructions in memory 406, ROM 408 or storage 410 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 400 may be coupled via I/O subsystem 402 to at least one output device 412. In one embodiment, output device 412 is a digital computer display. Examples of a display that may be used in various embodiments include a touch screen display or a light-emitting diode (LED) display or a liquid crystal display (LCD) or an e-paper display. Computer system 800 may include other type(s) of output devices 412, alternatively or in addition to a display device. Examples of other output devices 412 include printers, ticket printers, plotters, projectors, sound cards or video cards, speakers, buzzers or piezoelectric devices or other audible devices, lamps or LED or LCD indicators, haptic devices, actuators, or servos.

At least one input device 414 is coupled to I/O subsystem 402 for communicating signals, data, command selections or gestures to processor 404. Examples of input devices 414 include touch screens, microphones, still and video digital cameras, alphanumeric and other keys, keypads, keyboards, graphics tablets, image scanners, joysticks, clocks, switches, buttons, dials, slides, and/or various types of sensors such as force sensors, motion sensors, heat sensors, accelerometers, gyroscopes, and inertial measurement unit (IMU) sensors and/or various types of transceivers such as wireless, such as cellular or Wi-Fi, radio frequency (RF) or infrared (IR) transceivers and Global Positioning System (GPS) transceivers.

Another type of input device is a control device 416, which may perform cursor control or other automated control functions such as navigation in a graphical interface on a display screen, alternatively or in addition to input functions. Control device 416 may be a touchpad, a mouse, a trackball, or cursor direction keys for communicating direction information and command selections to processor 404 and for controlling cursor movement on display. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 414 may include a combination of multiple different input devices, such as a video camera and a depth sensor.

In another embodiment, computer system 400 may comprise an interne of things (IoT) device in which one or more of the output device 412, input device 414, and control device 416 are omitted. Or, in such an embodiment, the input device 414 may comprise one or more cameras, motion detectors, thermometers, microphones, seismic detectors, other sensors or detectors, measurement devices or encoders and the output device 412 may comprise a special-purpose display such as a single-line LED or LCD display, one or more indicators, a display panel, a meter, a valve, a solenoid, an actuator or a servo.

When computer system 400 is a mobile computing device, input device 414 may comprise a global positioning system (GPS) receiver coupled to a GPS module that is capable of triangulating to a plurality of GPS satellites, determining and generating geo-location or position data such as latitude-longitude values for a geophysical location of the computer system 400. Output device 412 may include hardware, software, firmware and interfaces for generating position reporting packets, notifications, pulse or heartbeat signals, or other recurring data transmissions that specify a position of the computer system 800, alone or in combination with other application-specific data, directed toward host 424 or server 430.

Computer system 400 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 400 in response to processor 404 executing at least one sequence of at least one instruction contained in main memory 406. Such instructions may be read into main memory 406 from another storage medium, such as storage 410. Execution of the sequences of instructions contained in main memory 406 causes processor 404 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage 410. Volatile media includes dynamic memory, such as memory 406. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 402. Transmission media can also take the form of acoustic or light waves, such as those generated during radio-wave and infra-red data communications.

Various forms of media may be involved in carrying at least one sequence of at least one instruction to processor 404 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 400 can receive the data on the communication link and convert the data to a format that can be read by computer system 400. For instance, a receiver such as a radio frequency antenna or an infrared detector can receive the data carried in a wireless or optical signal and appropriate circuitry can provide the data to I/O subsystem 402 such as place the data on a bus. I/O subsystem 402 carries the data to memory 406, from which processor 404 retrieves and executes the instructions. The instructions received by memory 406 may optionally be stored on storage 410 either before or after execution by processor 404.

Computer system 400 also includes a communication interface 418 coupled to bus 402. Communication interface 418 provides a two-way data communication coupling to network link(s) 420 that are directly or indirectly connected to at least one communication networks, such as a network 422 or a public or private cloud on the Internet. For example, communication interface 418 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 422 broadly represents a local area network (LAN), wide-area network (WAN), campus network, internetwork, or any combination thereof. Communication interface 418 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 418 sends and receives electrical, electromagnetic, or optical signals over signal paths that carry digital data streams representing various types of information.

Network link 420 typically provides electrical, electromagnetic, or optical data communication directly or through at least one network to other data devices, using, for example, satellite, cellular, Wi-Fi, or BLUETOOTH technology. For example, network link 820 may provide a connection through a network 422 to a host computer 424.

Furthermore, network link 420 may provide a connection through network 422 or to other computing devices via internetworking devices and/or computers that are operated by an Internet Service Provider (ISP) 426. ISP 426 provides data communication services through a world-wide packet data communication network represented as internet 428. A server computer 430 may be coupled to internet 428. Server 430 broadly represents any computer, data center, virtual machine, or virtual computing instance with or without a hypervisor, or computer executing a containerized program system such as DOCKER or KUBERNETES. Server 430 may represent an electronic digital service that is implemented using more than one computer or instance and that is accessed and used by transmitting web services requests, uniform resource locator (URL) strings with parameters in HTTP payloads, API calls, app services calls, or other service calls. Computer system 400 and server 430 may form elements of a distributed computing system that includes other computers, a processing cluster, server farm or other organization of computers that cooperate to perform tasks or execute applications or services. Server 430 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 830 may comprise a web application server that hosts a presentation layer, application layer and data storage layer such as a relational database system using structured query language (SQL) or no SQL, an object store, a graph database, a flat file system or other data storage.

Computer system 400 can send messages and receive data and instructions, including program code, through the network(s), network link 420 and communication interface 818. In the Internet example, a server 430 might transmit a requested code for an application program through Internet 428, ISP 826, local network 422 and communication interface 418. The received code may be executed by processor 804 as it is received, and/or stored in storage 410, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 404. While each processor 404 or core of the processor executes a single task at a time, computer system 400 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

Software Architectural Aspect

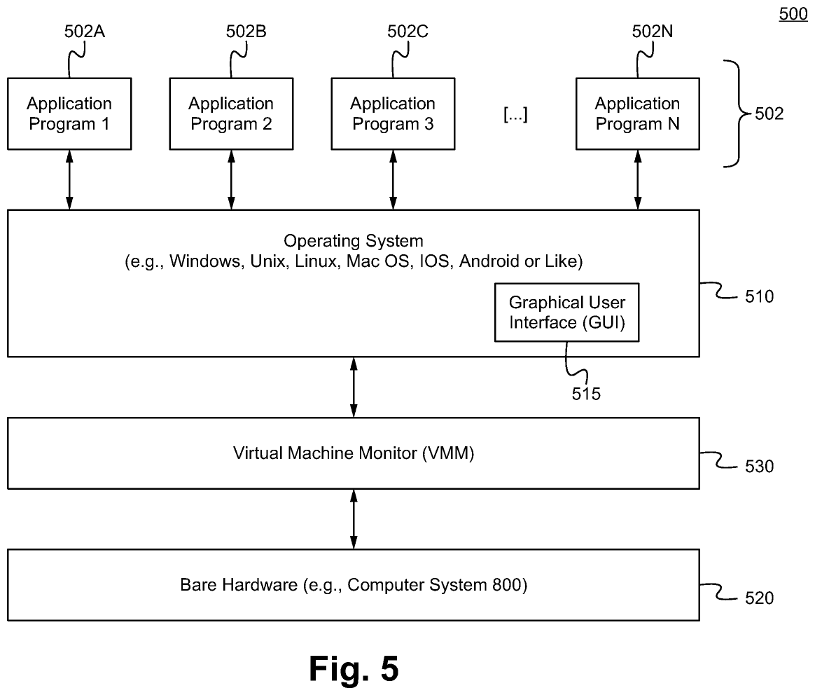

FIG. 5 is a block diagram of a basic software system 500 that may be employed for controlling the operation of computing device 500 in accordance with some embodiments. Software system 500 and its components, including their connections, relationships, and functions, is meant to be exemplary only, and not meant to limit implementations of the example embodiment(s). Other software systems suitable for implementing the example embodiment(s) may have different components, including components with different connections, relationships, and functions.

Software system 500 is provided for directing the operation of computing device 500. Software system 500, which may be stored in system memory (RAM) 506 and on fixed storage (e.g., hard disk or flash memory) 510, includes a kernel or operating system (OS) 510.

The OS 510 manages low-level aspects of computer operation, including managing execution of processes, memory allocation, file input and output (I/O), and device I/O. One or more application programs, represented as 502A, 502B, 502C . . . 502N, may be “loaded” (e.g., transferred from fixed storage 410 into memory 406) for execution by the system 500. The applications or other software intended for use on device 500 may also be stored as a set of downloadable computer-executable instructions, for example, for downloading and installation from an Internet location (e.g., a Web server, an app store, or other online service).

Software system 500 includes a graphical user interface (GUI) 515, for receiving user commands and data in a graphical (e.g., “point-and-click” or “touch gesture”) fashion. These inputs, in turn, may be acted upon by the system 500 in accordance with instructions from operating system 510 and/or application(s) 502. The GUI 515 also serves to display the results of operation from the OS 510 and application(s) 502, whereupon the user may supply additional inputs or terminate the session (e.g., log off).

OS 510 can execute directly on the bare hardware 520 (e.g., processor(s) 404) of device 400. Alternatively, a hypervisor or virtual machine monitor (VMM) 530 may be interposed between the bare hardware 520 and the OS 510. In this configuration, VMM 530 acts as a software “cushion” or virtualization layer between the OS 510 and the bare hardware 520 of the device 400.

VMM 530 instantiates and runs one or more virtual machine instances (“guest machines”). Each guest machine comprises a “guest” operating system, such as OS 510, and one or more applications, such as application(s) 502, designed to execute on the guest operating system. The VMM 530 presents the guest operating systems with a virtual operating platform and manages the execution of the guest operating systems.

In some instances, the VMM 530 may allow a guest operating system to run as if it is running on the bare hardware 520 of device 400 directly. In these instances, the same version of the guest operating system configured to execute on the bare hardware 520 directly may also execute on VMM 530 without modification or reconfiguration. In other words, VMM 530 may provide full hardware and CPU virtualization to a guest operating system in some instances.

In other instances, a guest operating system may be specially designed or configured to execute on VMM 530 for efficiency. In these instances, the guest operating system is “aware” that it executes on a virtual machine monitor. In other words, VMM 530 may provide para-virtualization to a guest operating system in some instances.

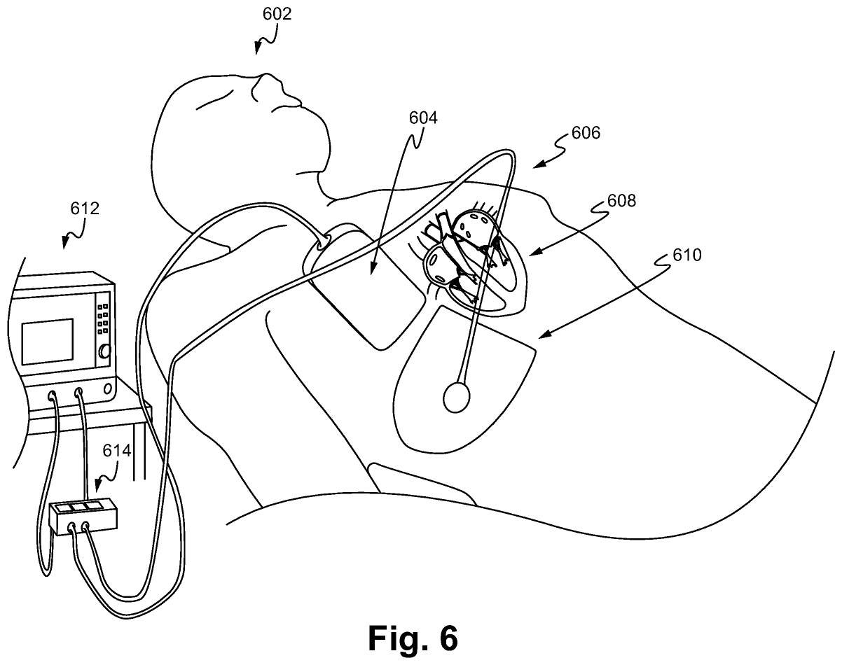

FIG. 6 illustrates a diagnostic and treatment system 600 in accordance with some embodiments. The system 600 includes a computing device 612 coupled with a controller 614, which are able to be configured to perform the functions, procedures, and tasks described herewithin (e.g, FIGS. 1-5), including performing various diagnostic, measuring and acquiring various bodily data, training machine learning and artificial intelligence models, refining and retraining the models, performing ablating under a predetermined condition among other treatment actions, performing after treatment diagnostics and/or adjust treatment plans.

In some embodiments, the system 600 performs bodily data collection on a patient/user 602 using various imaging or measuring devices 604, including ultrasound images, tissue voltage maps, CT scans, electroanatomic maps, MRI scans and metabolic maps from the MRI merged to give a predictive composite anatomy map 610 and target.

In some embodiments, the system 600 perform ablation at the predetermined site of an organ (e.g., a heart 608) with a predetermined dose of a radiofrequency using an ablation device 606. Radiofrequency ablation (RFA), also called fulguration, is a medical procedure in which part of the electrical conduction system of the heart, tumor or other dysfunctional tissue is ablated using the heat generated from medium frequency alternating current (in the range of 350-500 kHz).

The above-described basic computer hardware and software is presented for purpose of illustrating the basic underlying computer components that may be employed for implementing the example embodiment(s). The example embodiment(s), however, are not necessarily limited to any particular computing environment or computing device configuration. Instead, the example embodiment(s) may be implemented in any type of system architecture or processing environment that one skilled in the art, in light of this disclosure, would understand as capable of supporting the features and functions of the example embodiment(s) presented herein.

In some embodiments, the term ‘Planning Target Volume’ refers to the Clinical Target Volume plus a margin to allow for geometric uncertainty for the target shape.

In utilization, these models disclosed herein can be used for therapeutic, treatment, and/or diagnostic purposes, including myocardial tissue and is ablation of arrhythmia. The methods and devices improve patient eligibility and efficacy of cardiac ablation non-invasively. In some embodiments, the models, software, and hardware are used for non-treatment and non-diagnostic functions, such as for teaching demonstration, and tissue engineering experiments (e.g., in-vitro or in-vivo).

In operation, the procedure of utilizing machine learning is able to standardize the accurate radio-surgical targeting and ablate to treat cardiac arrhythmias.

I claim:

1. A method of identifying a myocardial target using a machine learning system including evaluating at least one characteristic of an unknow myocardial volume and an origin of an arrhythmia contained therein, or a combination thereof using a computer comprising:
a) constructing a transformation function mode and predicting at least one of unknown physiologic characteristic of at least one of a training myocardial tissue, a training electroanatomic mapping, or a training computerized imaging data set; and
b) performing a production mode by using the transformation function mode and the at least one of unknown physiologic characteristic to predict at least one of unknown anatomic characteristics or the unknow myocardial volume containing an arrhythmogenic foci.

2. The method of the claim 1, further comprising storing at least one feature vectors of a known anatomic characteristic of at least one production myocardial volume and an electrophysiologic footprint.

3. The method of the claim 2, further comprising calculating a dose and a volume of deposited radiation of an effective ablation.

4. The method of the claim 2, further comprising storing at least one feature vectors associated with a myocardial volume and an arrhythmia.

5. The method of the claim 2, further comprising perturbing at least one patient known characteristic or physiologic characteristic of at least one production myocardial volume and an arrhythmia location stored in at least one of the feature vectors.

6. The method of the claim 5, further comprising calculating a new approximate volume and arrythmia with the perturbed at least one patient known anatomic characteristic.

7. The method of the claim 5, further comprising storing quantities associated with the unknow myocardial volume planned for ablation in the at least one feature vectors.

8. The method of the claim 7, further comprising repeating the perturbing and the storing the least one feature vectors and the vectors associated with the unknow myocardial volume or an arrhythmia signal.

9. The method of the claim 1, further comprising applying the transformation function mode to one or more of feature vectors using a production mode.

10. The method of the claim 9, further comprising generating one or more quantities of interest with the production mode.

11. The method of the claim 10, further comprising storing the one or more quantities of interest with the production mode.

12. The method of the claim 11, further comprising processing, using the production mode, the quantities of interest to provide data for use in at least one of evaluation, diagnosis, prognosis, risk management, treatment and treatment planning related to at least one production myocardial volume or arrhythmia signal.

13. The method of the claim 12, further comprising using data for at least one of (1) guiding clinical decision-making, (2) providing predictive information about disease progression, (3) providing information for risk stratification, (4) providing for patient monitoring, (5) conducting sensitivity analyses, (6) evaluating an anatomic scenario, (7) evaluating an electrophysiologic or arrhythmia scenario, (8) estimating response to ablation, and (9) developing and understanding cardiac health and its relationship to arrhythmia.

14. A machine learning system configured to identify a myocardial target by evaluating at least one characteristic of an unknow myocardial volume and an origin of an arrhythmia contained therein, or a combination thereof, wherein the machine learning system comprising:
a) a transformation function mode predicting at least one of unknown physiologic characteristic of at least one of a training myocardial tissue, a training electroanatomic mapping, or a training computerized imaging data set; and
b) a production mode applying the transformation function mode to the at least one of unknown physiologic characteristic to predict at least one of unknown anatomic characteristics or the unknow myocardial volume containing an arrhythmogenic foci.

15. The machine learning system of the claim 14, further comprising a computed tomography device.

16. The machine learning system of the claim 14, further comprising a magnetic resonance imaging device or a positron emission tomography system.

17. The machine learning system of the claim 14, further comprising an ultrasound imaging device.

18. The machine learning system of the claim 14, further comprising a Doppler device.

19. The machine learning system of the claim 14, further comprising an electrophysiologic device.

20. The machine learning system of the claim 14, further comprising clinical instruments, catheters, intracavitary or intravascular monitoring system that measures parameters related to electrical signals, impedance, volume, flow, or pressure measurements.

21. The machine learning system of the claim 14, further comprising a radiation oncology treatment plan with inputs of does, volume, Planning Target Volume (PTV), conformality and other measures characteristically found in the radiation oncology plan.

22. The machine learning system of the claim 14, wherein the production mode is configured to process quantities of interest to provide data for use in at least one of evaluation, diagnosis, prognosis, risk, treatment and treatment planning related to at least one of a production myocardial volume or an arrhythmia signal.

23. The machine learning system of the claim 14, wherein the production mode provides data to be used in at least one of construction and execution of a computer-based model of at least one of myocardial volume and electrophysiologic signal or arrhythmia.

24. The machine learning system of the claim 14, further comprising a training mode configured to compute and construct the transformation function mode based on a plurality of images to predict the unknown anatomic myocardial volume.

25. The machine learning system of the claim 14, wherein the transformation function mode is based upon a least one morphologic simplification that exploits underlying myocardial geometry, electrophysiologic signals, changes in cellular metabolism, transmission of myocardial electrical signals, and tissue changes that correspond to changes in oxygenation and other physiologic parameters that influence the generation of aberrant rhythms and finally arrhythmia, with consequences to cardiac function.

26. A method of performing a non-invasive cardiac radiosurgery comprising:
a) identifying an arrhythmia abnormality by combining at least two of myocardial imaging data, metabolic data, and electrophysiologic data by a computer; and
b) predicting an effective therapeutic intervention of the arrhythmia abnormality by determining a dose, a location, a volume of a tissue to be treated or a combination thereof.

27. The method of claim 26, further comprising ablating the location with an effective radiofrequency and the dose.

28. The method of claim 26, wherein the myocardial imaging data are acquired from a computed tomography (CT), magnetic resonance imaging (MRI), or ultrasound imaging.

29. The method of claim 26, wherein the electrophysiologic data comprise tissue voltage maps or electroanatomic maps.

30. The method of claim 26, wherein the metabolic data are determined based on percentage of scared myocardium and its inherent electrical membrane channels.

31. The method of claim 26, further comprising using machine learning program to combine the at least two of the myocardial imaging data, the metabolic data, and the electrophysiologic data and to predict the effective therapeutic intervention of the arrhythmia abnormality.

32. The method of claim 31, wherein the machine learning program comprises a training mode, a transformation function mode, and a production mode.

33. The method of claim 32, wherein the training mode is configured to compute and store in a feature vector of one or more known characteristics.

34. The method of claim 32, wherein the training mode is further configured to repeat a set of perturbations and calculate and store steps to create a feature vector and volume, and to generate the transformation function mode.

35. The method of claim 34, wherein the production mode is configured to apply the transformation function mode to the feature vector.

36. The method of claim 26, further comprising radio-ablating an area of predicted susceptible to an arrhythmia generation.

37. The method of claim 36, wherein the area of predicted susceptible to the arrhythmia generation comprises an intersection area of tissues of late activation and tissue of fibrosis.

38. The method of claim 26, further comprising performing the steps a) and b) again to produce a revised treatment plan that treats the same or a near-by area to block the arrhythmia at an arrhythmia recurrence clinical event.
